(19) United States
(12) Patent Application Publication
(10) Pub. No.: US 2006/0213503 A1
(43) Pub. Date: Sep. 28, 2006

(54) Inhalation therapy device that can be actuated in different modes

(75) Inventors: Markus Borgschulte, Muenchen (DE); Andreas Pfichner, Unterhaching (DE)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Pari GmbH Spezialisten fuer effektive inhalation, Starnberg (DE)

(21) Appl. No.: 11/345,906

(22) Filed: Feb. 2, 2006

(30) Foreign Application Priority Data
Feb. 7, 2005 (DE) 10 2005 005 540.0

(51) Int. Cl. A61M 11/00

(52) U.S. Cl. 128/200.14

(57) ABSTRACT

The present invention relates to an inhalation therapy device having a control device which actuates an aerosol generating means in different modes simultaneously. With the inhalation therapy device according to the invention it is possible to generate specific aerosols relative to the predominant aspect of the therapy. The changeover between different aerosols is to be accomplished without any major effort by the patient and/or the therapist carrying out the treatment. The inhalation therapy device comprises, for the provision of a medicament in the form of an aerosol for inhalation with a nebulizing device, a membrane, an actuating device, which is designed such that it causes the membrane of the nebulizing device to oscillate, and a control device, designed such that it controls the actuating device in a first mode and in a second mode, whereby during actuation in the first mode, the membrane is actuated with a first working frequency, and during actuation in the second mode, the membrane is actuated with a second working frequency.

### FIELD OF THE INVENTION

The present invention relates to an inhalation therapy device, and in particular to an inhalation therapy device having a control device that actuates an aerosol generating means in different modes simultaneously.

### PRIOR ART

Inhalation devices are known which have membranes that are caused to oscillate by means of an actuating device so that an aerosol is generated with the help of the oscillating membrane from a liquid containing a medicament, said aerosol being presented to a patient for inhalation.

DE 101 22 065 A1, for example, describes an inhalation therapy device in which an aerosol is generated by a membrane that is caused to oscillate. In this case, the membrane is disposed on a substrate. Furthermore, an actuating device in the form of a piezoelectric element is provided, which is likewise attached to the substrate. The piezoelectric element is actuated and deflects as a function of an applied voltage. By actuating it with a high frequency, the piezoelectric element causes the substrate or membrane to oscillate. A fluid supplied to the membrane is nebulized by the oscillation of the membrane and is dispensed as an aerosol. This aerosol is mixed with the air inhaled by the patient and thus reaches the intended areas of the respiratory tract.

Furthermore, DE 101 22 065 A1 describes a control means of the actuating device with a frequency that is variable within a narrow range for locating a resonant operating frequency. As membrane nebulizers are frequently battery-operated, it is desirable to have an operating method which is as energy-saving as possible. This may be achieved, inter alia, by actuating the membranes at their resonant frequency. However, the resonant frequency depends significantly on the geometry of the oscillator, which consists of the membrane with the fluid provided for nebulization disposed thereon. The resonant frequency shifts by varying the fluid, for example by reducing the quantity during nebulization or by a change in temperature. However, so that the membrane can be operated at a resonant frequency in spite of this, the frequency is systematically varied within a narrow frequency range in which the resonant frequency is assumed to be, so as to determine the optimum operating frequency, i.e. the resonant frequency.

The membrane of an aerosol generator frequently has a perforated structure so that a fluid to be nebulized can be introduced from the rear of the membrane, said fluid then being dispensed through the apertures of the perforated structure of the membrane as an aerosol on the other side of said membrane when the membrane oscillates.

The aerosol dispensed by the membrane has a specific droplet spectrum which characterizes the dispensed aerosol in respect of the average droplet size and the distribution of the droplet size. The droplet spectrum is defined by the position of the maximum and by the distribution.

The droplet size is decisive for the site at which the aerosol is deposited. Large droplets are relatively heavy and sluggish and consequently do not follow the inhaled airflow in the respiratory tract but rather soon collide, as a result of their sluggishness, with the walls of the respiratory tract instead of following a curve of the respiratory tract, and are deposited there, for example on the mucous membranes of the mouth and throat. In contrast, smaller droplets tend to follow the inhaled airflow to a greater extent and reach deeper and narrower regions of the respiratory tract and are deposited there, i.e. they settle there. However, if the droplets are too small, they may not be deposited at all. They then leave the respiratory tract upon exhalation and are thus lost without being effective for the therapy. Furthermore, for certain medical conditions it is necessary for the medicament to not be deposited in the deepest regions of the respiratory tract, for example in the alveoli, but perhaps as soon as in the bronchi. For applications of this type, a somewhat larger droplet size, for example, is necessary.

The deposition site of the aerosol thus substantially depends on the geometry of the respiratory tract and the droplet size or droplet spectrum of the aerosol. The geometry of the respiratory tract is patient-specific and covers a huge range from adults to children and infants.

Applications of a medicament at a desired deposition site in the respiratory tract of the patient, the respiration characteristics of the patient and other therapy-related aspects make it seem desirable to have available an inhalation therapy device which generates different aerosols. Specific aerosols can then be generated relative to the predominant aspect of the therapy. It should thereby be possible for the patient and/or therapist carrying out the treatment to accomplish the changeover between different aerosols without any major effort.

Aerosols differ, for example, in respect of their droplet spectrum, i.e. the distribution of the quantity of droplets of differing sizes. Various measuring methods exist for determining the droplet spectrum or the parameters describing droplet distribution, such as the mass median diameter (MMD) for example. The droplet spectrum, expressed, for example, by the MMD value, is thus suitable as a reference parameter for differentiating between two aerosols generated by one and the same inhalation therapy device.

The aim of the invention is to provide an inhalation therapy device which allows generation of aerosols with at least two different droplet spectrums without major effort.

This object is solved by an inhalation therapy device for the provision of a medicament in the form of an aerosol for inhalation with a nebulizing device, having a membrane, an actuating device, which is designed such that it causes the membrane of the nebulizing device to oscillate, and a control device, designed such that it actuates the actuating device in a first mode and in a second mode, wherein during actuation in the first mode, actuation of the membrane is effected with a first working frequency, and during actuation in the second mode, actuation of the membrane is effected with a second working frequency.

Since, according to the invention, the actuating device causes the membrane to oscillate at different working frequencies by means of appropriate actuation by the control device, the aerosol is generated in such a varied manner that it is possible to set different droplet spectra. Excitation at different working frequencies alone is sufficient to do this. Unlike known solutions, two or more working frequencies (operating frequencies) are provided in the inhalation therapy device according to the invention, which each lead to different droplet spectra.

Although only a single membrane is provided in the inhalation therapy device, it is thus possible by means of actuation with two different working frequencies f₁ and f₂ in a first mode or a second mode to provide a first or a second droplet spectrum. In this case, the first droplet spectrum, for example, may be designed for the respiratory tract geometry of an adult whilst the second droplet spectrum is designed for the respiratory tract geometry of an infant. Alternatively, the droplet spectrum of the first mode may be designed for therapy in the upper respiratory tract whilst the droplet spectrum of a second mode may be designed for therapy of the lower respiratory tract. It is thus possible with a single inhalation therapy device, simply by means of a different actuation of different membrane areas or by actuation in different modes of the membrane, to provide different droplet spectra for different application purposes.

The generation of aerosols differing as regards the droplet spectrum can be specifically supported according to a first embodiment of the invention in that areas of the membrane, which are caused to oscillate to a specific extent at the different working frequencies, are provided with holes of differing sizes and/or distribution.

It is thus possible to achieve that in a first mode, by means of an area which is excited in said first mode, a droplet spectrum is attained that differs from a droplet spectrum which is generated in a second mode by an area of the membrane that is excited in a second mode. By exciting different areas in the different modes, it is thus possible to generate different droplet spectra with a different hole size or hole distribution, which can then be used for different therapy purposes without having to make significant structural alterations to the inhalation therapy device.

Alternatively, a fluid to be nebulized can also be introduced from the front of the membrane and dispensed as an aerosol from the same side of the membrane when said membrane oscillated. In this case, it is not imperative to have holes in the membrane. In fact, it is possible in this case to beneficially influence aerosol generation by means of a surface structure. However, a surface structure can also be advantageous for a membrane with holes, so that if, for example, a fluid is introduced to the rear of the membrane and an aerosol is generated through the holes, any fluids collecting at the front can be subsequently nebulized. A surface structure can be, in particular, an accumulation of crests or troughs of varying geometries and sizes, for example, cubes, cuboids, pyramids, spheres or mixtures thereof.

According to a further advantageous embodiment, in areas of the membrane that are caused to oscillate to a specific extent at the different working frequencies, the inhalation therapy device has a greater hole density or surface structure density in areas with a higher oscillatory deflection than in areas with a lower oscillatory deflection.

At points of the membrane which experience high deflection, the generation of an aerosol is especially effective if a larger number of holes or surface structures is present. In areas of lower deflection or at oscillation nodes which experience no deflection at all, it cannot be anticipated that an aerosol will be generated even if holes or structures are present. Due to the fact that in a conventional manufacturing process every hole in the membrane is made individually, it is also possible for reasons of efficiency to dispense with the holes at points with slight or absolutely no generation of aerosol. The same applies to surface structures. Furthermore, by dispensing with holes and structures at oscillation nodes or oscillation nodal lines which do not contribute to aerosol generation, it is possible to prevent fluid from passing through the membrane at that point by way of holes or to prevent liquid from inadvertently collecting on the structures. Such fluid which has not been nebulized leads to the formation of large drops on the membrane, which wet the membrane and can make further nebulization difficult or even prevent it.

The generation of aerosols differing as regards the droplet spectrum can be specifically supported according to a further embodiment of the invention by providing areas of the membrane, which are caused to oscillate to a specific extent at the different working frequencies, with a different curvature of the surface, or by providing a surface curvature of an area excited in the first mode which is different to that of an area excited in the second mode.

By means of modified radii of curvature in different areas of the membrane, it is possible to locally emphasize the individual oscillation modes such that if they are accordingly excited, certain areas oscillate in a more pronounced manner and make a greater contribution to the droplet spectrum than other areas.

The generation of aerosols differing as regards the droplet spectrum can be specifically supported according to a third embodiment of the invention by designing areas of the membrane with varying thicknesses, which are caused to oscillate to a specific extent at the different working frequencies.

By designing the membrane with areas having varying thicknesses, the basic oscillation behavior of the membrane areas alters as a function of the thickness. Thus, it is also possible, by appropriately selecting the membrane thickness, to promote or even suppress oscillation at a predetermined frequency in a specific area if no oscillation or only slight oscillation is desired in this area in a specific mode.

The generation of aerosols differing as regards the droplet spectrum can be specifically supported according to a further embodiment of the invention by designing areas of the membrane, which are caused to oscillate to a specific extent at the different working frequencies, in accordance with a combination of the peculiarities mentioned above.

According to a further advantageous embodiment of the invention, the working frequencies substantially correspond to a resonant frequency of the membrane or of one of the areas of the membrane or to a harmonic of the resonant frequency.

The loss of an oscillator is at a minimum in the case of resonance and the membrane caused to oscillate requires a lower amount of energy to oscillate. A lower energy requirement is always important if there is only a limited amount of energy available, for example in the case of a battery-operated inhalation therapy device. Furthermore, by emphasizing different resonances in different areas of the membrane, it is possible to ensure that the areas not oscillating in the resonant frequency attenuate themselves and thus contribute less to aerosol generation. As a result, a droplet spectrum generated by these areas can be reduced as compared to a droplet spectrum which is generated by an area oscillating at the resonant frequency. The adjustment or retention of a resonant working frequency may be carried out in a known manner, e.g. in accordance with DE 101 22 065 A1.

According to a further embodiment of the present invention, those areas of the membrane that are caused to oscillate to a specific extent at the different working frequencies bend upon actuation such that the curvature of the surface changes during said actuation.

If the curvature of the surface changes, the membrane bends in itself. As a result of such a bending of the membrane, the deflected areas in particular are active for aerosol generation, so that formation of the aerosol droplets and their release from the holes or apertures is promoted when the membrane oscillates.

According to a further embodiment of the present invention, upon excitation in at least one of the first or second modes, excitation of the other of the first or second mode is substantially less or essentially does not take place.

As a result, it is basically possible to ensure that in a first mode, which causes a first area to oscillate, the droplet spectrum generated by this area is more pronounced than that of another area, which is excited to a lesser extent or is not excited at all if operation is carried out in the first mode.

According to a further embodiment of the present invention, during excitation in the first mode one area oscillates substantially in the bending or curving mode and another area oscillates substantially in the displacement or deflection mode.

If oscillation takes place in the deflection mode, the formation of the droplets changes as compared to oscillation in the bending mode. In the bending mode, the membrane oscillates in itself, as a result of which droplet generation becomes more efficient, whereas in the deflection mode, the membrane is shifted as a whole. The droplet spectrum can also be adjusted in this manner.

According to a further embodiment of the present invention, an area oscillating in the first mode and an area oscillating in the second mode are disposed concentrically.

According to a further embodiment of the present invention, a medicament supply device is provided, which introduces a medicament from the side of the membrane facing away from the side on which an aerosol is generated and released from.

Thus, a fluid is introduced in such a manner that neither the fluid nor a supply device stands in the way of aerosol release.

According to a further embodiment of the present invention, an area oscillating in the first mode has a different internal mechanical stress to an area oscillating in the second mode.

An internal mechanical stress in a component changes the oscillation behavior of said component. By selectively introducing different pre-stresses into different areas of the membrane, it is possible to influence the oscillation behavior of different areas of said membrane.

According to a further embodiment of the present invention, the areas of the membrane which are caused to oscillate to a specific extent at the various working frequencies are delimited by an area which has a substantially higher curvature of the surface than the areas of the membrane which are caused to oscillate to a specific extent at the various working frequencies. Furthermore, the area may be designed such that an oscillation node is disposed in the area with the substantially higher curvature of the surface upon actuation of the membrane.

Such an area of substantially higher curvature may be, for example, a fold or a groove. It is possible to ensure that an oscillation node forms specifically at this point by appropriately shaping this area. Thus, if the surface of the membrane is suitably designed, it is possible to delimit an area with a specific oscillation behavior from other areas. It is also possible in this manner to introduce pre-stresses in this area, which likewise influence the oscillation behavior.

According to a further embodiment of the present invention, more than two modes, i.e. working frequencies, can also be provided.

By providing three or more modes, it is possible to make three or more different droplet spectra available, which open up an even broader area of use for the inhalation therapy device according to the invention.

According to a further embodiment of the present invention, several modes may be actuated simultaneously.

A broader area of use and a greater diversity of droplet spectra are likewise provided by a combination of several modes.

The present invention and its embodiments are explained by means of the following figures, with the figures merely serving to aid comprehension and in no way restricting the subject matter protected by the claims in light of the description and the figures.

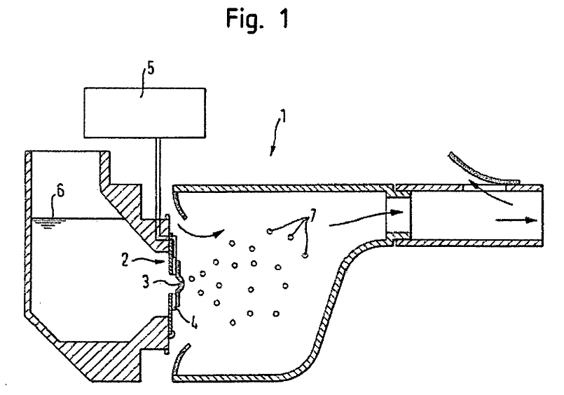

FIG. 1 shows a schematic arrangement of an inhalation therapy device;

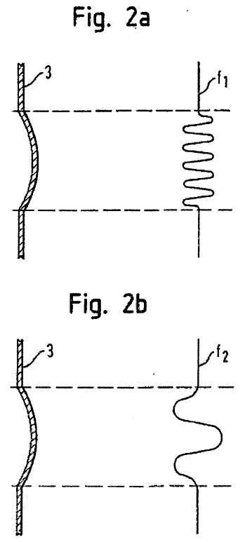

FIG. 2a shows a schematic representation of a membrane with a basic oscillation pattern in a first mode;

FIG. 2b shows a schematic representation of a membrane with a basic oscillation pattern in a second mode;

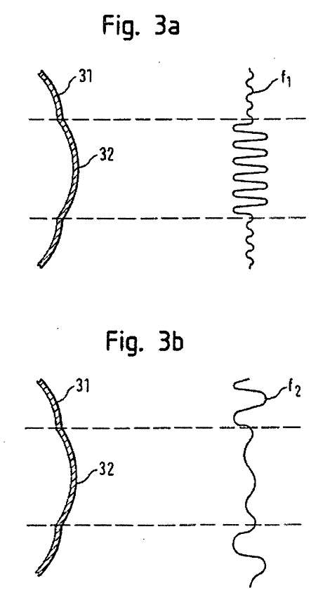

FIG. 3a schematically shows a membrane with increased oscillation in a first area in a first mode;

FIG. 3b schematically shows a membrane with increased oscillation in a second area in a second mode;

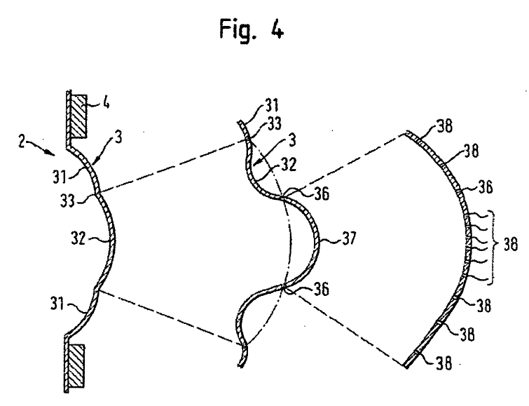

FIG. 4 shows a membrane with two areas and an oscillation arising in the first area as well as a hole distribution in a sub-area of the first area;

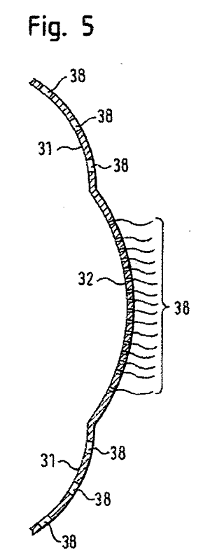

FIG. 5 shows a hole arrangement with respect to hole sizes and hole distribution in a first area; and furthermore a hole arrangement with respect to a hole size and a hole distribution in a second area;

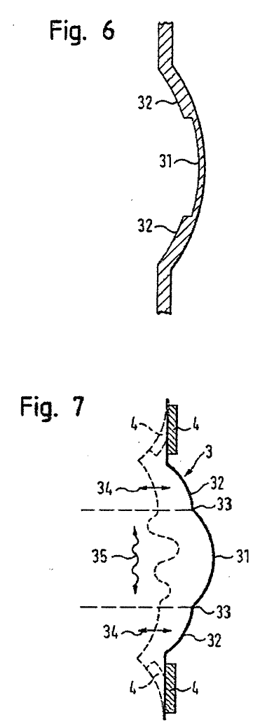

FIG. 6 shows a membrane with several areas which have different material thicknesses;

FIG. 7 shows an excitation of the membrane in such a way that a first area works in a or curving mode and a second area works in a deflection mode.

FIG. 1 shows a schematic arrangement of an inhalation therapy device 1 with a nebulizer device 2 having a membrane 3. The membrane 3 is linked with an actuating device 4. The membrane is disposed such that a fluid 6 is present on the membrane at the rear so that upon actuation of the membrane, the fluid present at the rear is dispensed as aerosol 7 through holes in the membrane (not shown in this figure). The actuating device 4 is linked with a controller 5 which is able to control the actuating device 4 such that the membrane is actuated in a first mode at a first working frequency and in a second mode at a second working frequency.

In this case, the working frequency is the frequency at which the membrane oscillates and dispenses a fluid 6 present at the rear as an aerosol 7 on the other side of the membrane 3. The membrane is only schematically represented in FIG. 1 in order to clarify the position in an inhalation therapy device 1 and to show the basic working principle of an inhalation therapy device with a membrane.

The aerosol 7 dispensed by the membrane is dispensed into a chamber in which the aerosol 7 mixes with the air present in said chamber so that the patient can inhale the air and aerosol mixture for therapy purposes. For reasons of clarity, all valves which are necessary for providing respiratory air and directing the flow of the inhaled or exhaled air have not been shown in this figure.

FIGS. 2a and 2b schematically show a membrane, with FIG. 2a showing an oscillation at the working frequency f₁, which represents a working frequency of the membrane in a first mode. FIG. 2b likewise shows a schematic representation of the membrane 3, however at a working frequency f₂, which occurs on the membrane by actuation in a second mode. Both representations are schematic and serve to explain the invention; it is not intended to reflect actual oscillation states.

The membrane 3 is caused to oscillate by the actuating device 4, which is not shown here, so that in a first mode it excites membrane 3 at a first working frequency f₁ and in a second mode it excites the membrane at a second working frequency f₂ which is different to working frequency f₁. As a result of the different working frequencies and the oscillation patterns adjusted on the membrane, the wave troughs and wave crests occur in different places at working frequency f₁ than at working frequency f₂, and thus membrane 3 differs from the oscillation pattern on the membrane surface at the different working frequencies f₁ and f₂. It is possible in this manner to generate aerosols with different droplet spectra.

As a result of the different oscillation patterns, other areas in one and the same membrane 3 deflect more strongly at working frequency f₁ than at working frequency f₂. This property can be used in order to provide different areas of the membrane with holes of different density and size, which can further assist a different droplet spectrum to arise during nebulization at working frequency f₁ than at working frequency f₂ since nebulization takes place through other areas of the membrane, and by reason of the fact that different hole geometries and hole densities are present at different points, this results in a different droplet spectrum arising.

FIG. 3a shows a membrane which has several areas with different radii of curvature. In this case, the membrane is designed such that during excitation with a working frequency f₁ in a first mode, a stronger oscillation forms in a first area 32 than in the second area 31. This can be achieved, for example, if at the working frequency f₁, the first area 32 has a resonant frequency or a harmonic of the resonant frequency so that upon excitation, these areas of the membrane are deflected more strongly. In a second area 31, the deflection is less when excited with the same working frequency f₁ since the resonant frequency of this area does not correspond to the working frequency f₁ or a harmonic thereof, and thus the deflection remains slight due to greater attenuation. Consequently, it is possible for the first area to be actuated and deflected more intensively when actuated with a first working frequency f₁ in a first mode so that the resultant droplet spectrum decisively depends on the oscillation and/or the supporting hole geometry and hole density in the first area of the membrane. In contrast hereto, FIG. 3b shows the same membrane which is, however, excited at a second working frequency f₂ in a second mode. However, the second working frequency f₂ is, for example, measured such that it corresponds to the resonant frequency of second area 31 or a harmonic thereof so that in comparison to the first area 32, a more pronounced oscillation arises at the second working frequency f₂ in the second mode due to the resonance conditions and less attenuation in second area 31. Thus, in the second mode at the second working frequency f₂, the droplet spectrum arising during nebulization is substantially dependent on the oscillation or the supporting hole geometry and hole distribution in the second area 31 of the membrane.

FIG. 4 shows a nebulizing device 2 having a membrane 3 and an actuating device 4, said membrane also having several areas 31, 32, 33 in this case too, on which various oscillations can develop when actuated in different modes. The first area 32 of the membrane is shown in FIG. 4 in the center and enlarged on the right, whereby in the first area 32 of the membrane an oscillation with three half-waves is schematically indicated, which has wave crests 37 and wave nodes 36. Due to the attachment of the membrane at the edge and an area 33 which delimits the first area 32 from the second area 31 and has a substantially greater surface curvature than the first and second areas, both the restraint of the membrane at the edge as well as the area with a substantially greater surface curvature 33 can be understood as fixed ends so that a standing wave forms between these fixed ends. This standing wave has wave nodes 36 at fixed points without it being necessary to clamp these node points tightly. Only the areas of wave troughs or wave crests 37 experience a strong deflection. A further section of the enlargement is in turn magnified, this magnified section showing a wave crest 37 between two wave nodes 36. Wave nodes 36 experience very slight or absolutely no deflection whereas wave crest 37 experiences strong deflection and strong deformation.

Generally speaking, aerosol generation only takes place at those points of a perforated membrane which experience strong deflection. Thus, according to an embodiment of the present invention, the holes are provided primarily in the areas of the membrane that experience strong deflection, i.e. wave crests 37. The areas that do not experience strong deflection, i.e. wave nodes 36, can consequently dispense with holes since nebulization would not take place even if holes were present at these points. To clarify this, FIG. 4 shows a greater hole density in the area of antipode 37, whereby the holes 38 are disposed very close to one another, whereas the distance increases in the direction of wave nodes 36 so the hole density on the membrane decreases in the direction of wave nodes 36. The production of holes in a membrane is relatively time-consuming and expensive using the methods currently known since every single hole has to be individually made in the membrane by means, for example, of a laser drill method. However, it is precisely the methods in which the holes are individually produced that are especially suitable for the application of the invention. This is because the manufacturing expenditure for producing a perforated membrane can turn out to be considerably lower if holes only have to be provided at points where nebulization can also actually take place.

According to a further embodiment of the present invention, the hole density varies in different areas of the membrane or the hole geometry varies in different areas of the membrane. FIG. 5 shows a membrane section in a first area 32 of the membrane 3 that is provided with holes 38, which, in FIG. 5a, have a relatively small diameter but which are, however, close together. FIG. 5 furthermore shows the hole distribution in a second area 31, in which the holes are relatively large as compared to the holes 38 of the first area but which are, however, further apart so that the hole density in this second area is smaller than in the first area 32. The combination of the hole diameter and the hole distribution can, however, also be reversed, i.e. larger holes with a wider spacing can be provided in the first area whilst smaller hole diameters with a narrower spacing can be provided in the second area 31. Likewise, holes with a small diameter but a wide spacing, i.e. a lower hole density, can be provided in one area and holes with a larger diameter and a wider spacing, i.e. a lower hole density, can be provided in another area so that at this point every combination of hole diameter and hole density is conceivable with an arrangement in different areas on the membrane. The positioning of the holes with appropriate hole diameters and hole distributions on appropriate areas of the membrane is incumbent on the person skilled in the art when designing an inhalation therapy device suitable for the application.

FIG. 6 shows a membrane which has a thinner material thickness in a first area than in a second area 32. The material thickness and thus the mass modify the oscillation behavior of the membrane or membrane areas so that due to a modified thickness of the membrane and to areas where the thickness changes abruptly, boundary conditions correspondingly arise for the oscillatable structure membrane and oscillation nodes can be expected at these transitions.

FIG. 7 shows a schematic representation of the membrane 3 with an actuating device 4, said membrane in turn having a first and second area. In this embodiment, the first area oscillates in a bending or curving mode 35 whereas the second area 34 oscillates in a deflection mode. An oscillation behavior such as this may arise due to external boundary conditions which are provided, for example, by a varying thickness or a varying geometric design of the membrane areas. Such an oscillation behavior may also arise due to varying resonant frequencies in the different areas of the membrane. It is also conceivable for the first area to move in a deflection mode and the second area to oscillate in a curving or bending mode. At this point too, different combinations are conceivable, which the person skilled in the art will select from his specialist knowledge in order to effect a suitable design of an inhalation therapy device.

1. Inhalation therapy device for the provision of a medicament in the form of an aerosol for inhalation, comprising
a nebulizing device having a membrane,
an actuating device designed in such a manner that it causes the membrane of the nebulizing device to oscillate, and
a control device designed in such a manner that it controls the actuating device in a first mode and in a second mode,
whereby during actuation in the first mode, the membrane is actuated with a first working frequency and during actuation in the second mode, the membrane is actuated with a second working frequency.

2. Inhalation therapy device according to claim 1, wherein in those areas of the membrane which are caused to oscillate to a specific extent at the different working frequencies, holes or surface structures of varying sizes and/or distribution are provided.

3. Inhalation therapy device according to claim 1, wherein in those areas of the membrane which are caused to oscillate to a specific extent at the different working frequencies, the hole density or the density of the surface structures is higher in areas of higher oscillatory deflection than in areas with lower oscillatory deflection.

4. Inhalation therapy device according to claim 1, wherein those different areas of the membrane which are caused to oscillate to a specific extent at the different working frequencies have different surface curvatures.

5. Inhalation therapy device according to claim 1, wherein a surface curvature of an area excited in the first mode and of an area excited in the second mode is different.

6. Inhalation therapy device according to claim 1, wherein in those areas of the membrane which are caused to oscillate to a specific extent at the different working frequencies, the membrane has varying thicknesses.

7. Inhalation therapy device according to claim 1, wherein the working frequencies substantially correspond to a resonant frequency of the membrane or one of the areas of the membrane or to a harmonic of the resonant frequency.

8. Inhalation therapy device according to claim 1, wherein those areas of the membrane which are caused to oscillate to a specific extent at the different working frequencies bend upon actuation so that the surface curvature changes upon actuation.

9. Inhalation therapy device according to claim 1, wherein upon excitation of an area in at least one of the first or the second modes, the excitation of an area of the other of the first or the second modes is considerably lower or essentially does not take place.

10. Inhalation therapy device according to claim 1, wherein upon excitation in the first mode, an area oscillates substantially in the bending or curving mode and another area oscillates substantially in the displacement or deflection mode.

11. Inhalation therapy device according to claim 1, wherein an area oscillating in the first mode and an area oscillating in the second mode are disposed concentrically.

12. Inhalation therapy device according to claim 1, wherein a medicament supplying device is provided, which supplies a medicament from the side of the membrane facing away from the side on which an aerosol is generated.

13. Inhalation therapy device according to claim 1, wherein an area oscillating in the first mode and an area oscillating in the second mode have a different internal mechanical stress.

14. Inhalation therapy device according to claim 1, wherein the areas of the membrane which are caused to oscillate to a specific extent at the different working frequencies are delimited by an area that has a substantially greater surface curvature than the areas of the membrane which are caused to oscillate to a specific extent at the different working frequencies.

15. Inhalation therapy device according to claim 14, wherein an oscillation node lies in the area with the substantially greater surface curvature upon actuation of the membrane.

16. Inhalation therapy device according to claim 1, wherein more than two modes are provided.

17. Inhalation therapy device according to claim 1, wherein several modes are actuated simultaneously.
